(19) United States
(12) Patent Application Publication
(10) Pub. No.: US 2003/0022864 A1
(43) Pub. Date: Jan. 30, 2003

(54) 9-[(5-dihydroxyboryl)-pentyl] purines, useful as an inhibitor of inflammatory cytokines

(76) Inventors: Khalid S. Ishaq, Chapel Hill, NC (US); George J. Cianciolo, Chapel Hill, NC (US)

Correspondence Address: JENKINS & WILSON, PA, 3100 TOWER BLVD, SUITE 1400, DURHAM, NC, 27707, US

(21) Appl. No.: 09/841,148

(22) Filed: Apr. 24, 2001

(51) Int. Cl. A61K 31/69; C07F 5/02

(52) U.S. Cl. 514/64; 544/229

(57) ABSTRACT

A compound of Formula I

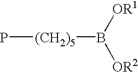

wherein R¹ and R² are both hydrogen atoms or R¹ and R² together are a propylene chain bridging the two oxygen atoms and P is a purine base residue bonded via the N⁹; and the pharmaceutically acceptable salts thereof.

### TECHNICAL FIELD

The present invention relates, in general, to novel N-substituted-(dihydroxyboryl)alkyl purines, which are useful as inhibitors of inflammatory cytokines, and more specifically, relates to 9-[(5-dihydroxyboryl)-pentyl] purines.

Table of Abbreviations

| | |
|---|---|
| AD | Alzheimer's disease |
| ACR2O | American College of Rheumatology Criteria |
| AIDS | acquired immune deficiency syndrome |
| BMMCs | bone marrow mast cells |
| BSA | bovine serum albumin |
| BHR | bronchial hyperresponsiveness |
| CCR1 | C-C chemokine receptor 1 |
| CCR4 | C-C chemokine receptor 4 |
| CCR5 | C-C chemokine receptor 5 |
| C | Centigrade |
| CNS | central nervous system |
| CSF | cerebrospinal fluid |
| CIA | collagen-induced arthritis |
| CD4 | common determinant 4 |
| CD8 | common determinant 8 |
| CHF | congestive (or chronic) heart failure |
| Cd | Crohn's disease |
| DMSO | dimethyl sulfoxide |
| DNP | dinitrophenyl |
| DMEM | Dulbecco's Minimum Essential Medium |
| ESR | erythrocyte sedimentation rate |
| EAE | experimental allergic encephalomyelitis |
| FBS | fetal bovine serum |
| GLUT | glucose transporter |
| GAPDH | glyceraldehyde-3-phosphate dehydrogenase |
| GM-CSF | granulocyte-macrophage colony stimulating |
| | factor |
| HDL | high density lipoprotein |
| HAART | highly active antiretroviral therapy |
| HHMC | human heart mast cells |
| HIV | human immunodeficiency virus |
| IgG | immunoglobulin G |
| IBD | inflammatory bowel disease |
| IC₅₀ | inhibitory concentration of the compound at |
| | which there is a 50% reduction in activity; the |
| | lower the IC₅₀ is, then the more potent the |
| | compound is |
| ICAM | intercellular adhesion molecule |
| IFN | interferon |
| IL | interleukin |
| kDa | kilo Dalton |
| kg | kilogram |
| LVEF | left ventricular ejection fraction |
| LD | lipodystrophy |
| LPS | lipopolysaccharide |
| LSM | lymphocyte separation medium |
| LAK | lymphokine activated killer |
| MCAP | macrophage chemotactic activating protein |
| mRNA | messenger ribonucleic acid |
| m | meter |
| μg | microgram |
| μl | microliter |
| mg | milligram |
| ml | milliliter |
| mM | millimolar |
| MEM | minimal essential medium |
| MS | multiple sclerosis |
| ng | nanogram |
| NK | natural killer |
| NYHA | New York Heart Association |
| NZW | New Zealand White |
| NIDDM | non-insulin-dependent diabetes mellitus |
| n | number |
| PBMC | peripheral blood mononuclear cells |
| PBS | phosphate buffered saline |
| PHA | phytohemagglutinin |
| PAI | plasminogen activator inhibitor |
| p24 Gag | name of a particular structural protein of the |
| | HIV-1 virus |
| PASI | psoriasis area and severity index |
| PsARC | Psoriatic Arthritis Response Criteria |
| RT-PCR | Reverse Transcriptase - Polymerase Chain |
| | Reaction |
| RA | rheumatoid arthritis |
| RPMI | Roswell Park Memorial Institute |
| AB serum | serum obtained from blood that is type AB |
| SMC | smooth muscle cell |
| SAK2 | name of a particular stimulatory |
| | oligonucleotide |
| TCID | tissue culture infectious dose |
| TF | tissue factor |
| TG | triglycerides |
| TNF-α | tumor necrosis factor alpha (also known as |
| | cachectin) |
| UC | ulcerative colitis |
| U | units |
| WHHL | Watanabe heritable hyperlipidemic |
| WEHI-3 | name of a cell culture cell line |

### BACKGROUND OF THE INVENTION

The most pertinent background art of which the present inventors are aware is U.S. Pat. No. 5,643,893, issued Jul. 1, 1997, to Benson et al. This patent discloses that certain N-substituted (dihydroxyboryl)alkyl purine derivatives, where the alkyl moiety was normal butyl, were made. These purine derivatives are somewhat similar to the inventive compounds of Formula I described below, but the inventive compounds have a normal pentyl moiety instead of a normal butyl moiety. Unexpectedly, the present inventors discovered that the inventive compounds with the pentyl moiety possess an increased activity, when tested in the assay for inhibition of TNF-α production by human monocytes, as compared to the compounds with the butyl moiety as made by Benson et al.

More generally in connection with the background art, the following is noted.

Tumor necrosis factor alpha (TNF-α), also known as cachectin, is a 17 kDa protein produced by monocytes, macrophages, activated lymphocytes, neutrophils, NK (natural killer) cells, LAK (lymphokine activated killer) cells, mast cells, astrocytes, adipocytes, endothelial cells, smooth muscle cells, and some transformed cells. A large number of studies reveal that TNF-α is produced principally by macrophages and that it may be produced in vitro as well as in vivo. TNF-α is a cytokine that mediates a wide variety of biological activities, including: cytotoxic effects against tumor cells, activation of neutrophils, growth proliferation of normal cells, enhancement of HIV (human immunodeficiency virus) viral replication, and immunoinflammatory, immunoregulatory, and antiviral responses. TNF-α also induces the secretion of interleukin-1 (IL-1) and is a primary mediator of inflammation and endotoxin-induced shock.

Also of interest, a 26 kDa membrane form of TNF-α has been described on the surface of monocytes and activated T-cells (i.e., the T-group of lymphocytes). This molecule may be involved in intracellular communication, as well as cytotoxic activity, and is a surface marker for lymphocyte activation. By a variety of techniques TNF-α has been shown to exist in a trimer in aqueous solutions; only a small fraction of human TNF-α molecules occur as monomers at physiological ionic pH.

Two distinct TNF-α receptors have been identified: a 55 kDa receptor and a 75 kDa receptor, TNFR-I and TNFR-II respectively. The intracellular domains of the two TNF-α receptor types are apparently unrelated, suggesting that they employ different signal transduction pathways. While both receptors are capable of binding TNF-α and activating the transcription factor NFκB, the expression of each receptor appears to be independently and differentially regulated. Human TNF-α will bind to both types of receptors with equal affinity on human cells.

TNF-α has been found to be an important mediator of the pathophysiological effects of a diverse array of invasive diseases, infections, and inflammatory states. As a consequence of the production (or overproduction) of TNF-α in tissues, and as a consequence of the presence of other cytokines in the cellular environment, TNF-α may ultimately benefit or injure the host.

For instance, when produced acutely and released in large quantities into the circulation during a serious bacterial infection, TNF-α triggers a state of shock and tissue injury (septic shock syndrome) that carries an extremely high mortality rate (30 to 90%). Three main lines of evidence indicate that TNF-α plays a central role in the development of septic shock: (1) administration of TNF-α to mammals induces a state of shock and tissue injury that is nearly indistinguishable from septic shock; (2) inhibition of TNF-α in septic shock prevents the development of both shock and tissue injury and confers a significant survival advantage; and (3) production of TNF-α occurs in animals and humans during experimental and clinical septic shock syndrome.

When produced during chronic disease states, TNF-α mediates cachexia, a syndrome characterized by anorexia, accelerated catabolism, weight loss, anemia, and depletion of body tissues. Weight loss frequently develops during chronic illness and, if not reversed, may kill the host before the underlying disease can be eradicated.

For instance, it is not unusual for the patient afflicted with cancer or AIDS to lose 50% of body weight and to succumb to complications of malnutrition. By contrast to starvation, during which protein-conserving adaptive responses are maximally operative, the cachectic host tends to catabolize body energy stores in the face of suppressed intake, thus hastening the host's own demise.

In addition to being implicated in septic shock and cachexia, TNF-α has been implicated in the pathophysiology of a number of additional diseases. These include, but aren't restricted to, rheumatoid arthritis (RA), inflammatory bowel disease (IBD) [i.e., ulcerative colitis (UC) or Crohn's disease (Cd)], multiple sclerosis (MS), congestive or chronic heart failure (CHF), psoriasis, asthma, non insulin-dependent diabetes mellitus (NIDDM), cerebral malaria, anemia associated with malaria, stroke, the development of Alzheimer's disease (AD), periodontitis, and the weight loss associated with AD, cancer, or AIDS.

In rheumatoid arthritis (RA), for instance, evidence exists of macrophage activation with demonstration of increased amounts of two monokines, TNF-α (i.e., TNF-α is the type of cytokine produced by monocytes and thus is a monokine) and IL-1, in the serum but even more in the synovial fluid. TNF-α, an inducer of IL-1, is significantly elevated in RA but not in reactive arthritis. Moreover, TNF-α levels in RA correlate with the synovial fluid leukocyte count and with the ESR (erythrocyte sedimentation rate). TNF-α is an important mediator of immunity and inflammation. Because of the biologic activities (activation of neutrophils, release of arachadonic acid metabolites from synovial cells, induction of cartilage resorption, inhibition of proteoglycan release in cartilage, and induction of macrophage chemotactic activating protein [MCAP]) possessed by TNF-α, it is one of the potential mediators in chronic arthritis.

Studies have shown that monoclonal antibody to TNF-α and soluble TNF-α receptors can ameliorate joint disease in murine collagen-induced arthritis (CIA). In these studies, anti-TNF-α or soluble TNF-α receptors administered prior to the onset of disease significantly reduced paw swelling and histological severity of arthritis or the level of circulating anti-type II collagen IgG. More relevant to human disease was the ability of the antibody or soluble receptor to reduce the clinical score, the paw swelling, and the histological severity of disease even when injected after the onset of clinical arthritis.

Both a humanized murine monoclonal antibody to TNF-α (REMICADE® the registered trademark of Centocor, Inc., which is located in Malvern, Pa., United States of America, and which is a wholly-owned subsidiary of Johnson & Johnson; REMICADE® is the trademark for infliximab) and a soluble human TNFR-II chimeric protein (ENBREL®, fusion protein with the Fc domain of human IgG₁; ENBREL® is the registered trademark of Immunex, Inc., which is located in Seattle, Wash.; ENBREL® is the trademark for etanercept) have undergone extensive human clinical trials and are approved for marketing for treatment of rheumatoid arthritis in the U.S. and elsewhere. Both of these products are TNF-α antagonists; i.e., they block the biological activity of TNF-α by binding to it and preventing it from exerting its biological effects.

Crohn's disease (Cd) and ulcerative colitis (UC) are chronic inflammatory bowel diseases of unknown etiology but circumstantial evidence exists that immune mechanisms may play an important role in the pathogenesis of the intestinal lesion and that cytokines produced by lymphoid cells may be critical for the extra-intestinal sequelae of the disease. In both Cd and UC, activation of macrophages seems to be a key feature and increased production of the macrophage-derived cytokines TNF-α, IL-1, and IL-6 have been reported in both diseases. A published study determined the location and tissue density of cells immunoreactive for TNF-α in intestinal specimens from 24 patients with chronic inflammatory bowel disease (15 with Cd, 9 with UC) and 11 controls. There was significantly increased density of TNF-α immunoreactive cells in the lamina propria of both Cd specimens and UC specimens suggesting that this degree of TNF-α production probably contributes significantly to the pathogenesis of both Cd and UC by impairing the integrity of epithelial and endothelial membranes, increasing inflammatory cell recruitment, and creating prothrombotic effects on the vascular endothelium.

Multiple sclerosis (MS) is a chronic, inflammatory, demyelinating disease of the central nervous system (CNS). The majority of infiltrating cells at the site of demyelination are macrophages and T-cells. IL-1 and TNF-α in the CSF are detected at higher levels and more frequently in patients with active MS than in patients with inactive MS or with other neurological diseases. In a study of MS patients, Beck and colleagues found an increase of TNF-α and interferon production by peripheral blood mononuclear cells two weeks prior to disease exacerbation. Experimental allergic encephalomyelitis (EAE) is the best characterized demyelinating disease of the CNS in animals. EAE and MS share many characteristics. Ruddle and colleagues used a monoclonal antibody that neutralizes TNF-α to treat EAE in mice (Ruddle et al., J. Exp. Med., 1990, 172:1193-1200). The incidence and severity of EAE in the antibody-treated mice were dramatically reduced and the onset of disease was delayed. Moreover, the authors reported that the preventive therapy was long-lived, extending through 5 months of observation.

Recent studies suggest that TNF-α has an important role in the pathogenesis of CHF. The evidence supporting the importance of TNF-α in heart failure stems from the following observations: (a) increased levels of circulating TNF-α occurred in patients with advanced heart failure; (b) patients who became symptomatic with respect to heart failure had progressively higher circulating TNF-α levels; and (c) when TNF-α was chronically administered to rodents, the animals developed a cardiomyopathy characterized by depressed systolic function. Furthermore, transgenic mice that overexpress TNF-α in the myocardium developed a cardiomyopathy and died prematurely, even in the absence of elevated TNF-α in the periphery.

Myocytes have been demonstrated to produce TNF-α in response to various stimuli, including pressure or volume overload. Furthermore, studies have shown that TNF-α is present in failing but not in non-failing myocardium. Support for the role of TNF-α in the pathogenesis of heart failure comes from several clinical studies. Pentoxifylline, a nonspecific agent that suppresses TNF-α production, was used in a double-blind, placebo-controlled study in 28 patients with symptomatic heart failure. The patients in the study were treated for 6 months with either pentoxifyllline (400 mg, 3 times daily; n=14) or placebo (n=14). At the end of the study, there were 4 deaths, all in the placebo group. There was an increase in left ventricular ejection fraction (LVEF) (38.7 versus 26.8%, p=0.04) and more patients were free of symptoms in the treated group than in the placebo control group.

Two additional clinical studies utilized ENBREL®), the much more specific TNF-α antagonist molecule. The first study was a placebo-controlled dose-finding and safety study. Several patients receiving ENBREL® had significant improvement in symptoms and exercise tolerance with a decrease in serum levels of biologically-active TNF-α by 85%. The second ENBREL® study was a double-blind, randomized, placebo-controlled study of two doses (5 and 12 mg/m², twice weekly) of ENBREL®. The study showed that ENBREL® administration for 3 months resulted in a trend toward overall improvements in NYHA (New York Heart Association) classification and quality of life, particularly in those receiving the higher dose.

The release of cytokines from cutaneous cells may be of major importance in the initiation and development of many inflammatory skin disorders. For example, TNF-α, which in healthy skin is found preformed only in mast cells, is able to induce the expression of several adhesion molecules including intercellular adhesion molecule-1 (ICAM-1). Increased expression of ICAM-1 occurs in keratinocytes in lesional skin of psoriasis and atopic dermatitis and is considered to be an important initiator of leukocyte/keratinocyte interactions.

The authors of a 1997 report (Mizutani et al., 1997, J. Derm. Sci., 14:145-53) looked at the spontaneous production of TNF-α, IL-1β, and IL-6 from the peripheral blood mononuclear cells (PBMC) of psoriasis patients. The production of all three inflammatory cytokines by psoriatic PBMC was significantly higher than that by normal PBMC. IL-1β and TNF-α showed a positive relation to clinical severity but IL-6 did not. TNF-α production increased much more than did the others.

Perhaps the first antedotal clinical evidence for a role of TNF-α in the pathogenesis of psoriasis was provided by a report (Oh et al., 2000, J. Amer. Acad. Derm., 45:829-30) of treatment of a woman (suffering from refractory IBD, a 15-year history of Crohn's disease, and a 20-year history of moderate to severe psoriasis) with a single infusion of the TNF-α antagonist REMICADE™. Two weeks after the infusion, the patient's psoriasis had dramatically cleared up.

ENBREL® was recently tested in a randomized, double-blind, placebo-controlled, 12-week study in 60 patients with psoriatic arthritis and psoriasis. In this study (Mease et al., 2000, The Lancet, 356:385-90) psoriatic arthritis endpoints included the proportion of patients who met the Psoriatic Arthritis Response Criteria (PsARC) and who met the American College of Rheumatology preliminary criteria for improvement (ARC20). Psoriasis endpoints included improvement in the psoriasis area and severity index (PASI) and improvement in prospectively-identified individual target lesions. In the 12-week study, 87% (26 patients) of the ENBREL®-treated patients met the PsARC compared to 23% (7 patients) of the placebo-controlled patients. The ARC20 was achieved by 73% (22 patients) of the ENBREL®-treated patients compared to 13% (4 patients) of the placebo-controlled patients. Of the 19 patients in each treatment group who could be assessed for psoriasis (3% body surface area), 26% (5 patients) of the ENBREL®-treated patients achieved a 75% improvement in the PASI, compared with 0 of the placebo-treated patients (p=0.015). The median PASI improvement was 46% in ENBREL®-treated patients versus 9% in placebo-treated patients; similarly, median target lesions were 50% and 0% respectively.

TNF-α is implicated in the pathogenesis of asthma. Asthma is associated with the presence of an inflammatory cell infiltrate in the bronchial mucosa consisting of activated mast cells, eosinophils, and T cells. Several cytokines are considered to play a critical role in this response, particularly IL-4, IL-5, IL-6, and TNF-α. Bradding et al. (Amer. J. Resp. Cell & Mol. Biol., 1994, 10:471-80) reported a 7-fold increase in the number of mast cells staining for TNF-α in bronchial mucosal biopsies from asthmatics. Asthma is a disease characterized by bronchial hyperresponsiveness (BHR). Although the underlying mechanisms that induce this increase in bronchial reactivity remain unknown, evidence suggests that the inflammatory process present in the airways could play an important role in the development of BHR. This may result from alterations in the intrinsic properties of airway smooth muscle induced by inflammatory mediators. TNF-α is one possible candidate since on one hand, TNF-α is able to induce, in humans and in animals, a BHR to different inhaled pharmacological agents, and on the other hand, high levels of TNF-α were found in asthmatic airways. A Phase II clinical study of ENBREL® therapy in a segmental allergen bronchprovocation model of atopic asthma is currently being conducted by the National Heart, Blood, and Lung Institute of the National Institutes of Health. The goal of this study is to assess whether inhibition of TNF-α bioactivity can attenuate airway inflammation in mild-to-moderate allergic asthmatics.

Obesity is associated with an increased incidence of insulin resistance, dyslipoproteinemia, and hypercoagulability. In a more recently established hypothesis of body weight control and regulation of metabolism, the adipocyte secretes leptin and locally expresses TNF-α, the latter being responsible for the expression of metabolic cardiovascular risk factors. TNF-α mRNA expression and TNF-α protein are greatly increased in adipose tissue from obese animals and humans. Elevated TNF-α expression induces insulin resistance by downregulating the tyrosine kinase activity of the insulin receptor and decreasing the expression of GLUT-4 glucose transporters. TNF-α also reduces lipoprotein lipase activity in white adipocytes, stimulates hepatic lipolysis, and increases plasminogen activator-1 (PAI-1) content in adipocytes. Thus recent studies examining the link between insulin resistance and the development of obesity and noninsulin-dependent diabetes mellitus are consistent with the involvement of TNF-α as a central mediator (reviewed by Qi and Pekala, 2000, Proc. Soc. Exp. Biol. Med., 223:128-35).

Inflammatory mechanisms and immune activation have been hypothesized to play a role in the pathogenesis of age-associated diseases such as dementia and atherosclerosis. Bruunsgaard et al. (J. Gerontol., Series A, Biol Sci. & Med. Sci., 1999, 54:M357-64) evaluated the plasma concentration of TNF-α in a large group of centenarians and examined possible associations of TNF-α with cognitive function, atherosclerosis, and general health status. Plasma TNF-α was measured in 126 centenarians, as well as in 45 subjects aged 81 years, 23 subjects aged 55-65 years, and 38 subjects aged 18-30 years. The concentration of TNF-α was significantly increased in the 126 centenarians compared to the younger control groups, and a high concentration of TNF-α was associated with both AD and generalized atherosclerosis in the centenarians.

There are a number of potential sources for elevated levels of TNF-α in atherosclerosis. For instance, Newman et al. (J. Surg. Res., 1998, 80:129-35) showed that coronary arteries and smooth muscle cells cultured from those vessels could be stimulated to produce levels of TNF-α that were nearly 50-fold above the background levels observed for unstimulated cells. Another potential cellular source of TNF-α are mast cells. Mast cells have been identified in human heart tissue in close proximity to the sarcolemma, in perivascular and adventitial locations, and in the shoulder region of coronary atheroma. Human heart mast cells (HHMC) contain TNF-α in secretory granules and mast cell density is increased in the hearts of patients with ischemic and idiopathic dilated cardiomyopathy. Previous ultrastructural and immunocytochemical studies have shown that macrophages as well as smooth muscle cells (SMCs) are constituents of atherosclerotic lesions in the Watanabe heritable hyperlipidemic (WHHL) rabbit. In a study by Lei and Buja, they had shown that TNF-α mRNA levels in aorta of 18-month-old WHHL rabbits were significantly higher than that of 6-month-old WHHL rabbits and New Zealand White (NZW) rabbits. In a later study (Atherosclerosis, 1996, 125:81-9), Lei and Buja showed that the TNF-α gene was expressed in the medial SMCs as well as cells of intimal lesions in arteries of WHHL rabbits. TNF-α protein was also detected in the cytoplasm of the intimal and medial SMCs and macrophages by immunocytochemistry. In contrast, the expression of TNF-α mRNA and protein was not detected in arteries from healthy NZW rabbits.

Proinflammatory cytokines such as TNF-α and IL-1 are upregulated within hours in ischemic brain lesions. Either directly or via induction of neurotoxic mediators such as nitric oxide, cytokines may contribute to infarct progression in the post-ischemic period. Meistrell et al. (Shock, 1997, 8:341-8) examined the potential role of TNF-α in cerebral ischemia using a standard model of permanent focal cortical ischemia in rats, in which the volume of cerebral infarction is measured after permanent occlusion of the middle cerebral artery. Administration of neutralizing anti-rat TNF-α antibodies (P114) into the brain cortex significantly reduced ischemic brain damage (85% reduced infarct volume as compared with pre-immune treated controls). Similar results were obtained by systemic administration of CNI-1493, a tetravalent guanylhydrazone compound, which effectively inhibited endogenous brain TNF-α synthesis and conferred significant protection against the development of cerebral infarction (80% reduced infarct volume as compared with vehicle controls treated 1 hour post ischemia with 10 mg/kg). P114 anti-TNF-α and CNI-1493 were each cerebroprotective when given within a clinically relevant time window for up to 2 hours after the onset of ischemia. Lavine et al (J. Cerebral Blood Flow Metab., 1998, 18:52-8) studied the effect of anti-TNF-α antibody in a rat model of reversible middle cerebral artery occlusion. During focal ischemia and early reperfusion, TNF-α was rapidly and transiently released into circulation. Pretreatment with intravenous anti-TNF-α antibody reduced cortical (71%, p<0.015) and subcortical (58%, p<0.007) injury, enhanced cerebral blood flow during reperfusion, and improved the neurological outcome. Studies such as these described above suggest that inhibiting TNF-α may represent a novel pharmacological strategy to treat ischemic stroke.

Periodontal disease is the most frequent cause of tooth loss in humans and is the most prevalent disease associated with bone loss, including osteoporosis. To assess the role of IL-1 and TNF-α in this process, Assuma et al. (J. Immunol., 1998, 160:403-9) conducted studies in a Macaca fascicularis primate model of experimental periodontitis. Function-blocking soluble receptors to IL-1 and TNF-α were applied by local injection to sites with induced periodontal destruction and compared with similar sites injected with vehicle alone. The results indicated that injection of soluble receptors to IL-1 and TNF-α inhibited by approximately 80% the recruitment of inflammatory cells in close proximity to bone. The formation of osteoclasts was reduced by 67% at the experimental sites compared with that of control sites, and the amount of bone loss was reduced by 60%. These findings indicated that a significant component of the pathological process of periodontitis was due to IL-1/TNF-α activity, since inhibiting IL-1/TNF-α reduced both inflammatory cell recruitment and bone loss.

A chronic inflammatory response, possibly mediated by beta amyloid protein, is believed to be a major factor in the pathology of AD. Beta amyloid peptide can, in a dose-dependent manner, induce TNF-α in the murine-derived J774 monocyte/macrophage cell line. Using an animal model, Sutton et al (J. Submicroscopic Cytol. Pathol., 1999, 31:313-23) investigated the role of TNF-α and IL-1 in the beta amyloid-induced inflammatory response. Adult male rats were perfused, via an intra-aortic cannula, with either beta amyloid alone, beta amyloid plus IL-1 receptor antagonist (IL-1ra), beta amyloid plus TNF-α binding protein (TNF-α bp), or saline alone. Serum analysis showed a significant increase in TNF-α and beta amyloid after the injection of beta amyloid but no significant increase in either IL-1 or nitric oxide (NO). In rats given beta amyloid alone there was extensive vascular disruption, including endothelial and smooth muscle cell damage with leukocyte adhesion and migration, of the mesenteric arterioles and venules. Animals receiving either IL-1ra or TNF-α bp before beta amyloid showed no in vivo leukocyte extravasation or vascular damage. Therefore, the cytokines TNF-α and IL-1 seem to mediate the vascular disruption and inflammatory response initiated by beta amyloid and antagonism of these cytokines may offer new avenues for AD therapy.

TNF-α has been suggested to play a role in both replication of human immunodeficiency Virus-1 (HIV-1) and in the pathogenesis associated with HAART (highly active antiretroviral therapy), and in particular, with therapies utilizing anti-protease drugs. TNF-α is also thought to be one of the main factors associated with HIV-induced cachexia. HIV-1 infection of human PBMC has been shown to elicit secretion of several different cytokines, including TNF-α. TNF-α secretion induced by this virus has been of particular interest because the secretion has been associated with the development of HIV-1 dementia and because TNF-α increases viral replication by enhancing NF-kappaB interaction with the viral promoter, the HIV-1 long terminal repeat. Thus, an autocrine pathway is potentially created in which HIV-1 stimulates its own replication. Khanna et al. (J. Immunol., 2000, 164:1408-15) recently examined the TNF-α-eliciting properties of primary and laboratory strains of HIV-1. Khanna et al. found that the relative TNF-α-inducing ability of different variants was conserved when using PBMC from different individuals. Elicitation of TNF-α secretion was not blocked by exposure of cells to zidovudine, indicating that viral integration was not required to induce secretion. Rather, Khanna et al. found that the interaction between the virus and the cell surface was critical for TNF-α induction, since antibodies against CD4 or CCR5, the two major receptors for HIV-1, blocked the induction of TNF-α synthesis by PBMC when added before virus exposure.

In addition to the potential role of TNF-α in enhancing HIV replication, TNF-α has been hypothesized to play a role in the lipodystrophy associated with HIV infection. HIV infection induces an early decrease of cholesterol and a late increase of triglycerides (TG) with a reduction of HDL (high density lipoprotein). Both the increase of TG synthesis and the decrease of TG catabolism, in conjunction with a reduction in lipoprotein lipase activity, are responsible for these changes. As TNF-α has been shown to reduce lipoprotein lipase activity in white adipocytes, TNF-α may be, at least in part, responsible for the lipodystrophy associated with HIV infection.

Furthermore, although HAART has led to a dramatic decrease in the morbidity of patients infected with HIV, metabolic side effects, including lipodystrophy-associated (LD-associated) dyslipidemia, have been reported in patients treated with antiretroviral therapy. For instance, Ledru et al. (Blood, 2000, 95:3191-8) reported on a study designed to determine if successful HAART was responsible for a dysregulation in the homeostasis of TNF-α, a cytokine known to be involved in lipid metabolism. Ledru et al. observed that LD is associated with a more dramatic TNF-α dysregulation and with positive correlations between the absolute number of TNF-α CD8 T-cell precursors and lipid parameters usually altered in LD, including cholesterol, triglycerides, and the atherogenic ratio apolipoprotein B (apoB)/apoA1. Accordingly, Ledru et al. suggested that the proinflammatory response induced by efficient antiretroviral therapy is a risk factor of LD development in HIV(+) patients.

### SUMMARY AND OBJECTS OF THE INVENTION

The present invention relates to novel N-substituted (dihydroxyboryl)alkyl purine derivatives which are useful as inhibitors of inflammatory cytokines and proteins such as TNF-α, GM-CSF and tissue factor (TF). More particularly, the present invention relates to novel inhibitors of inflammatory cytokines and proteins, which inhibitors are compounds of Formula I

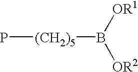

wherein R¹ and R² are both hydrogen atoms or R¹ and R² together are a 3 to 5 alkylene chain (such as a propylene chain, a butylene chain, or a pentylene chain) bridging the two oxygen atoms and P is a purine base residue bonded via the N⁹; and the pharmaceutically acceptable salts thereof.

Preferably P is selected from the group consisting of adenine, guanine, xanthine, and hypoxanthine and 8-substituted-, 6-substituted- or 2,6-disubstituted-purines wherein the substituents are selected from the group consisting of halogen and amino.

When both R¹ and R² are H, then Formula I is referred to below as Formula Ia. When R¹ and R² together form propylene bridging the two oxygens, then Formula I is referred to below as Formula Ib.

It is thus an object of the present invention to provide N-substituted-(dihydroxyboryl)alkyl purine derivatives which, by virtue of their ability to inhibit inflammatory cytokines, are useful as therapeutic agents for the treatment of invasive diseases, infections, and inflammatory states, particularly septic shock, rheumatoid arthritis, osteoarthritis, ulcerative colitis, Crohn's disease, psoriatic arthritis, psoriasis, chronic or congestive heart failure, asthma, multiple sclerosis, non insulin-dependent diabetes mellitus, atherosclerosis, multiple myeloma, neurological disorders (e.g., neurological ischemia and Alzheimer's disease), periodontitis, malaria or cerebral malaria, AIDS, and cachexia associated with HIV infection, cancer, or infection.

It is further an object of the present invention to provide synthetic procedures for the preparation of the novel N-substituted-(dihydroxyboryl)alkyl purine derivatives.

It is a still further object of the present invention to provide a method for treating a warm-blooded vertebrate animal, for instance a bird or a mammal, particularly a human, affected with septic shock, rheumatoid arthritis, osteoarthritis, ulcerative colitis, Crohn's disease, psoriatic arthritis, psoriasis, chronic heart failure, congestive heart failure, asthma, multiple sclerosis, non insulin-dependent diabetes mellitus, atherosclerosis, multiple myeloma, neurological disorders (e.g., neurological ischemia and Alzheimer's disease), periodontitis, malaria, cerebral malaria, AIDS, cachexia associated with HIV infection, cachexia associated with cancer, and/or cachexia associated with infection, which comprises the administration of an agent which is an inhibitor of inflammatory cytokines, namely an agent that comprises a N-substituted-(dihydroxyboryl)alkyl purine derivative.

It is thus a further object of the present invention to provide an AIDS therapy which, in addition to decreasing cachexia, decreases viral load and decreases lipid dystrophy associated with HIV infection or antiretroviral therapy by administration of a N-substituted-(dihydroxyboryl)alkyl purine derivative.

It is a still further object of the present invention to provide a therapeutic agent, namely a N-substituted-(dihydroxyboryl)alkyl purine derivative, which treats a warm-blooded vertebrate animal, for instance a bird or a mammal, affected with septic shock, rheumatoid arthritis, osteoarthritis, ulcerative colitis, Crohn's disease, psoriatic arthritis, psoriasis, chronic heart failure, congestive heart failure, asthma, multiple sclerosis, non insulin-dependent diabetes mellitus, atherosclerosis, multiple myeloma, neurological disorders (e.g., neurological ischemia and Alzheimer's disease), periodontitis, malaria, cerebral malaria, AIDS, cachexia associated with HIV infection, cachexia associated with cancer, and/or cachexia associated with infection by inhibiting Tumor Necrosis Factor (TNF) and/or by inhibiting other inflammatory cytokines which are mediators of these diseases.

Yet another aspect of the present invention provides a pharmaceutical formulation comprising a compound of Formula I and one or more pharmaceutically acceptable carriers, excipients or diluents.

It is an advantage of the present invention to provide a compound with far better pharmacological potency than those compounds made in the above-noted U.S. Pat. No. 5,643,893.

Some of the objects of the invention having been stated, other objects, as well as other advantages, will become evident as the description proceeds in connection with the Figures and the Laboratory Examples below.

### BRIEF DESCRIPTION OF THE DRAWINGS

FIG. 1 is a graph of the results of 2-amino-6-chloro-9-[(5-dihydroxyboryl)-pentyl] purine, labeled as LMP-420, tested for its effects on TNF-α production by a murine macrophage cell line.

FIG. 2 is a graph of the results of tests showing that this same compound, 2-amino-6-chloro-9-[(5-dihydroxyboryl)-pentyl] purine, had no effect on the production of nitric oxide as measured by the release of nitrate or nitrite.

FIG. 3 is a graph of the results of 2-amino-6-chloro-9-[(5-dihydroxyboryl)-pentyl] purine, labeled as LMP-420, tested for its effects on TNF-α production by a murine mast cell line.

FIG. 4 is a graph of the results of 2-amino-6-chloro-9-[(5-dihydroxyboryl)-pentyl] purine, labeled as LMP-420, tested for its effects on serum TNF-α production in mice infected with Plasmodium chabaudi.

FIG. 5 is a graph of the results of 2-amino-6-chloro-9-[(5-dihydroxyboryl)-pentyl] purine, labeled as LMP-420, tested for its effects on replication of HIV-1_{Bal} (Bal is a particular strain of HIV-1) in human peripheral blood mononuclear cells.

FIG. 6 is a photograph of a chromatography experiment that shows incubation of normal human PBMC with LMP-420 resulted in a dose-dependent inhibition of the expression of mRNA encoding for CCR1 as determined by RT-PCR analysis.

### DETAILED DESCRIPTION OF THE INVENTION

In the purine base structure, the conventional numbering of the substituents is as shown below:

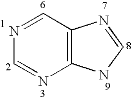

The purine base portion of the compounds of Formula I is bonded to the remainder of the structure through the nitrogen atom at the nine-position (N⁹).

In the compounds of Formula I, the purine base residue can be derived from the naturally occurring purine bases, such as adenine and guanine. The term purine base refers to these bases and analogs thereof, such as derivatives comprising alkyl, aralkyl, halogen, acetyl, hydroxymethyl, amido, and/or carbamate substituents. Accordingly, the dihydroxyboryl bases of the invention may be derived from a naturally occurring base, such as adenine, guanine, xanthine, or hypoxanthine (the latter two being natural degradation products), or from various chemically synthesized analogs thereof known in the art.

Certain illustrative 6-substituted and 2,6-disubstituted purine derivatives are those described as starting materials for the final products described in U.S. Pat. No. 4,199,574, the disclosure of which is incorporated by reference. These purines have the formula

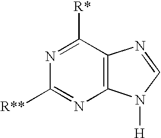

wherein R* is hydrogen, halogen, hydroxy, alkoxy, azido, thio, alkylthio, amino, alkylamino, or dialkylamino; and R** is hydrogen, halogen, alkylthio, acylamino, amino or azido. In this formula, halogen includes fluorine, chlorine, bromine and iodine, and alkyl groups contain 1 to 6 carbon atoms and acyl groups contain 2 to 7 carbon atoms. It is also contemplated for the present invention that one or both of R* or R** could be aroylthio and the purine could be 8-substituted with R* or R**.

Other useful purine bases are those described in Volumes I-III of “Nucleic Acid Chemistry”, ed. By Leroy B. Townsend and R. Stuart Gibson, Wiley Interscience.

In the practice of the instant invention, preferred purine bases are selected from the group consisting of 6-chloropurine, 2-amino-6-chloropurine, adenine, guanine, xanthine and hypoxanthine. Of these, 2-amino-6-chloropurine is a highly preferred purine base for use in preparing compounds of the instant invention.

Thus, preferred compounds of Formula I are the dihydroxyboryl-purine derivatives wherein the purine base portion is derived from of 6-chloropurine, 2-amino-6-chloropurine, adenine, guanine, xanthine and hypoxanthine.

Equivalent to the compounds of Formula I are biocompatible and pharmaceutically acceptable salts thereof.

Treatment of warm-blooded vertebrate animals

Contemplated is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the treatment of birds, including the treatment of those kinds of birds that are endangered, kept in zoos, as well as fowl, and more particularly domesticated fowl, i.e., poultry, such as turkeys, chickens, ducks, geese, guinea fowl, emus, and the like, as they are also of economical importance to humans. Thus, contemplated is the treatment of livestock, including, but not limited to, domesticated swine (pigs and hogs), ruminants, horses, poultry, and the like.

More particularly, a treatment effective amount of the inventive compound of Formula I is administered to the warm-blooded vertebrate animal. Thus, the invention comprises administration of the compound of Formula I in concentrations calculated to provide the animal being treated with the appropriate milieu to provide an effect of inhibition of inflammatory cytokines.

Medical conditions that can be treated include but are not limited to septic shock, rheumatoid arthritis, osteoarthritis, ulcerative colitis, Crohn's disease, psoriatic arthritis, psoriasis, chronic heart failure, congestive heart failure, asthma, multiple sclerosis, non insulin-dependent diabetes mellitus, atherosclerosis, multiple myeloma, neurological disorders (e.g., neurological ischemia and Alzheimer's disease), periodontitis, malaria, cerebral malaria, AIDS, cachexia associated with HIV infection, cachexia associated with cancer, and/or cachexia associated with infection. Representative neurological disorders are alos disclosed in U.S. Pat. Nos. 6,177,077 and 6,015,557, the disclosure of each of which is herein incorporated by reference in its entirety.

The compound of Formula I may be administered to the animal as a suppository or as a supplement to fluids that are administered enterally or parenterally, for instance nutriment fluids such as intervenous sucrose solutions. Furthermore, intraoral (such as buccal or sublingual) administration or transdermal (such as with a skin patch) administration to the animal is also contemplated. A good discussion of intraoral administration can be seen in U.S. Pat. No. 4,229,447 issued Oct. 21, 1980 to Porter and U.S. Pat. No. 5,504,086 issued Apr. 2, 1996 to Ellinwood and Gupta. A good discussion of transdermal administration can be seen in U.S. Pat. No. 5,016,652 issued May 21, 1991 to Rose and Jarvik.

Additionally, administration to the animal may be by various oral methods, for instance as a tablet, capsule, or powder (crystalline form) that is swallowed. Also, oral administration may include that the compound of Formula I is admixed in a carrier fluid appropriate therefor so that it is administered as a liquid (solution or suspension) orally. When the compound of Formula I is admixed in a carrier fluid, appropriate fluids include, but are not limited to, water, rehydration solutions (i.e., water with electrolytes such as potassium citrate and sodium chloride, for instance the solution available under the trade name RESOL® from Wyeth Laboratories), nutritional fluids (i.e., milk, fruit juice), and combinations thereof. Thus, the oral administration may be as a component of the diet, such as human food, animal feed, and combinations thereof.

In addition to oral administration such as by way of the mouth, contemplated also is administration of a solution or suspension to the esophagus, stomach, and/or duodenum, such as by the enteral administration method known as gavage, i.e., by way of a feeding tube. Gavage type of administration is useful for when the animal is very ill and can no longer swallow food, medicine, et cetera, by mouth.

Hence, it is also contemplated that additional ingredients, such as various excipients, carriers, surfactants, nutriments, and the like, as well as various medicaments, other than the compound of Formula I, may be present, whatever the form that the compound of Formula I is in. Medicaments other than the compound of Formula I may include, but are not limited to, osmolytic polyols and osmolytic amino acids (i.e., myo-inositol, sorbitol, glycine, alanine, glutamine, glutamate, aspartate, proline, and taurine), cardiotonics (i.e., glycocyamine), analgesics, antibiotics, electrolytes (i.e., organic or mineral electrolytes such as salts), and combinations thereof.

A suitable dosing amount of the compound of Formula I for administration to the animal should range from about 0.5 mg to about 7.0 mg per kg of body weight of the animal per day, more preferably from about 1.5 mg to about 6.0 mg per kg of body weight of the animal per day, and even more preferably from about 2.0 mg to about 5.0 mg per kilogram of body weight of the animal per day. Administration may be one or more times per day to achieve the total desired daily dose. Of course, the amount can vary depending on the severity of the illness and/or the age of the animal.

The present invention indicates that the compounds of Formula I process the ability to inhibit inflammatory cytokines.

Manufacture of compounds of Formula I

The novel compounds of Formula I can be produced by the synthetic pathways shown in Schemes I and II below.

In Scheme I, the synthetic process provides for the preparation of the compounds of Formula I wherein R¹ and R² are both hydrogen atoms. Scheme I is as follows.

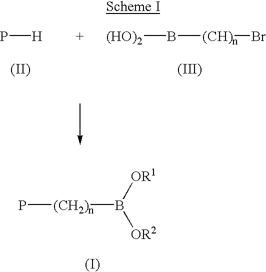

wherein R¹ and R² are both hydrogen atoms, P is hereinabove defined, and n=5.

In reaction Scheme I, the purine base of formula II was reacted with the dihydroxyborylalkyl bromide of Formula II, in the presence of a base and an acid acceptor, to afford the compounds of Formula I wherein R¹ and R² are both hydrogen atoms. Typically, the base is an inorganic base, such as potassium carbonate or sodium hydride, with the acid acceptor being potassium carbonate. Reaction times vary from 12 to 48 hours, and usual reaction temperatures are at room temperature.

In Scheme II, the synthetic process provides for the preparation of the compounds of Formula I wherein R¹ and R² together are a propylene chain bridging the two oxygen atoms. Scheme II is as follows.

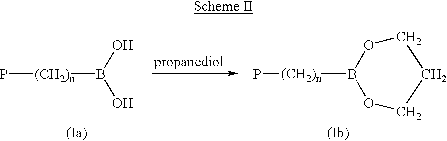

In reaction Scheme II, if a compound of Formula Ia, wherein both R¹ and R² are hydrogens, P is as hereinbefore defined, and n=5, is reacted with propanediol in a polar, anhydrous solvent, such as tetrahydrofuran, then provided are the desired compounds of Formula Ib wherein R¹ and R² together are a propylene chain bridging the two oxygen atoms. Typically, this reaction is conducted for periods of about 4-16 hours, and at room temperature. Of course, it is also contemplated that butanediol could be used (and then R¹ and R² are a butylene chain) or pentanediol could be used (and then R¹ and R² are a pentylene chain).

The starting dihydroxyboryl alkyl bromide of Formula III was conveniently prepared by reaction of a gamma-bromo-1-alkene with catecholborane. Typically, this reaction is conducted under a nitrogen atmosphere for a period of time of about 2-6 hours and a temperature of about 80° to 100° C., followed by aqueous hydrolysis to obtain the desired product. If desired, this starting dihydroxyboryl alkyl bromide of formula III can be recrystallized from chloroform.

The utility of compounds of Formula I can be demonstrated by activity in standardized assays, described in the Laboratory Examples and Figures below.

### Laboratory Examples

Assay for inhibition of TNF-α production by human monocytes

Monocytes were prepared by centrifugal counterflow elutriation from PBMC obtained from leukophoresis of normal volunteers (leukopaks) at the Phoresis laboratory located at Duke University Hospital, Durham, N.C., United States of America. PBMC from leukopaks were diluted in sterile isotonic saline/10 mM HEPES (N-[2-Hydroxyethyl]piperazine-N′-[2-ethanesulfonic acid]), the name of a buffer used in biological and protein chemistry) and placed into 50 ml conical polypropylene tubes in 30 ml aliquots. Each aliquot of diluted PBMC is underlaid with 20-25 ml of sterile lymphocyte separation medium (LSM; Organon-Technika, Durham, N.C., United States of America). The tubes were centrifuged at 400× gravity for 40 minutes at room temperature. The mononuclear cells at the interface were resuspended in phosphate buffered saline (PBS) and then separated, using a Beckman elutriator, into lymphocytes and monocytes. Yields of 10⁹ monocytes with greater than 90% purity were routinely obtained.

Purified monocytes prepared as described above were suspended at 4×10⁶ cells/ml in complete medium. Complete medium is defined as follows: RPMI-1640 supplemented with 100 U/ml penicillin; 100 μg/ml streptomycin; 2 mM L-glutamine; 1 mM Na pyruvate; 1% MEM non-essential amino acids; 25 mM HEPES (all from GIBCO, Gaithersburg, Md., United States of America); and 5% pooled, heat-inactivated (56° C., 30 minutes) human serum from type AB blood (Pel-Freez, Brown Deer, Wis., United States of America). To each well of a 48-well flat bottomed tissue culture plate was added 0.125 ml of cell suspension. Test materials (diluted in complete medium at 2× the desired final concentration) were added in 250 μl volumes to each well. Control wells received 250 μl of complete medium. All samples were tested at a minimum of 4 concentrations in the presence or absence of 100 ng/ml LPS (S. typhosa; Sigma, St. Louis, Mo., United States of America; 125 μl of 4× desired final concentration added) and incubated at 37° C. in humidified 5% CO₂ for 16 hours. At this time, culture supernatants were aspirated off and the unattached cells and cell debris removed by a 2 minute spin in a microcentrifuge at 10,000× gravity. The release of TNF-α was determined in the cell-free supernatants using an ELISA capture assay.

Assay for inhibition of TNF-α or nitric oxide (NO) by a murine macrophage cell line

Murine macrophage cell line J774.1 cells were maintained by culture at 37° C. in 5% humidified CO₂ in media that was Dulbecco's Minimum Essential Media (DMEM) supplemented with 100 U/ml penicillin; 100 μg/ml streptomycin; 2 mM L-glutamine; 1 mM Na pyruvate; 1% MEM non-essential amino acids; 25 mM HEPES (all from GIBCO, Gaithersburg, Md., United States of America); and 10% pooled, heat-inactivated (56° C., 30 minutes) fetal bovine serum (FBS; GIBCO). For assays of TNF-α and nitric oxide production, cells were cultured at 1×10⁵ cells/well in 96-well tissue culture plates. Test compound was prepared as a stock solution in DMSO and added to each well to give the desired final concentration. DMSO was added to control wells to serve as a vehicle control. Each concentration of test material or vehicle control was tested in replicates of 4 wells. After a 1 hour exposure to test material, a stimulus (either SAK2 stimulatory oligonucleotide [10 μg] or LPS [10 ng/ml]+IFNγ[100 u/ml]) was added to each well, and the plates incubated for 48 hours at 37° C. in 5% humidified CO₂. The supernatant media of each well were removed and tested for either TNF-α by ELISA or for nitric oxide products (nitrate/nitrite) using the Griess reagent as previously described (Weinberg, J. B. et al, 1994, J. Exp. Med., 179:651-60). Content of nitrite/nitrate in J774.1 cell culture supernatants was expressed as μM.

Assay for inhibition of TNF-α by a murine mast cell line

Bone marrow mast cells (BMMCs) were cultured from stem cells from the bone marrow of BALB/c mice as described (Malaviya, R. and Abraham, S. N., 1995, Methods Enzymol., 253:27-43). The cells were grown in 25% WEHI-3 conditioned medium and used for experiments after 20 days in culture. Mast cells harvested from such cultures are generally >98% pure, as determined by toluidine blue staining, and resemble mucosal-type mast cells. Monolayers of mast cells in 96-well tissue culture plates in serum-free RPMI-1640 medium containing 15 mM HEPES were sensitized with anti-dinitrophenyl (DNP) IgE (1:100 dilution; Sigma) for 4 hours at 37° C. Test compound was added for an additional hour at 37° C. The cells were washed 3 times to remove excess IgE. Anti-DNP-BSA (100 ng/ml; Sigma) was added for an additional 5 hours and supernatants were collected and assayed for TNF-α by a standard cytotoxicity assay (Malaviya et al, 1996, Nature [London], 381:77-80).

Assay for inhibition of HIV-1 replication

Human PBMC were cultured at 500,000 cells/well in 96-well tissue culture plates in the presence of 5 μg/ml PHA (phytohemagglutin-P; Sigma) and IL-2 (interleukin-2). Test compound was added to the PBMC at 3 hours prior to the addition of virus (HIV-1, Bal strain) at a TCID₅₀ of 500 (tissue culture infective dose). After an overnight incubation at 37° C., the cells were washed twice with growth medium and the growth medium was replaced with fresh growth medium containing the initial concentration of test compound. Each day a sample of the culture supernatants from the untreated but viral-infected wells was removed and assayed for the presence of HIV-1 p24 by solid phase ELISA. When the amount of p24 in the untreated but viral infected wells reached a level at which a 10-fold reduction could be readily determined (usually 5-6 days) the experiment was terminated and the concentration of p24 determined for each of the wells, both treated and untreated.

Tests

Representative compounds of the below Formula were tested in the above described assay for inhibition of TNF-α production by human monocytes, and the results were as shown below in Table 1.

Representative compounds of the below Formula were

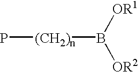

and the representative compounds were Comparison Compounds 1-17 when R¹ and R² were both hydrogen atoms or a propylene chain bridging the two oxygen atoms; P was a purine base residue bonded via the N⁹ or N⁷; and n was 3, 4, or 6; or the purine was bonded via the N⁷ and n was 5; and the pharmaceutically acceptable salts thereof. However, the representative compound was of Formula I when R¹ and R² were both hydrogen atoms; P was a purine base residue bonded via the N⁹; and n was 5; and the pharmaceutically acceptable salts thereof.

TABLE 1

| Comparison | | | | |
|---|---|---|---|---|
| Compounds | | | | IC₅₀ Against |
| # | Ring | Ring Substitution | Side-Chain | TNF-α (nM) |
| 1 | purine | 2-amino-6-chloro | 9-(CH₂)₄B(OH)₂ | 800 |
| 2 | purine | 6-chloro | 9-(CH₂)₄B(OH)₂ | 2500 |
| 3 | purine | 2-amino-6-chloro | 7-(CH₂)₄B(OH)₂ | 3000 |
| 4 | purine | 2-amino-6-oxo | 9-(CH₂)₄B(OH)₂ | 11000 |
| 5 | purine | 6-oxo | 9-(CH₂)₄B(OH)₂ | >50000 |
| 6 | purine | 2-amino-6-chloro | 9-(CH₂)₃CH₂OH | >50000 |
| 7 | purine | 2,6-dichloro | 9-(CH₂)₄B(OH)₂ | 800 |
| 8 | purine | 2-acetamido-6-chloro | 9-(CH₂)₄B(OH)₂ | 8000 |
| 9 | purine | 6-amino | 9-(CH₂)₄B(OH)₂ | 1000 |
| 10 | purine | 2,6-diamino | 9-(CH₂)₄B(OH)₂ | 2500 |
| 11 | purine | 6-methyl | 9-(CH₂)₄B(OH)₂ | 1500 |
| 12 | purine | 6-aminomethyl | 9-(CH₂)₄B(OH)₂ | >20000 |
| 13 | purine | 2-amino-6-chloro | 9-(CH₂)₃CO₂H | >20000 |
| 14 | purine | 2-amino-6-chloro | 9-(CH₂)₃CO₂CH₂CH₃ | >20000 |
| 15 | purine | 2-propylamido-6-chloro | 9-(CH₂)₄B(OH)₂ | 10000 |
| 16 | purine | 2-amino-6-chloro | 7-(CH₂)₅B(OH)₂ | 900 |
| 17 | purine | 2-amino-6-chloro | 9-(CH₂)₆B(OH)₂ | 1000 |
| Inventive | purine | 2-amino-6-chloro | 9-(CH₂)₅B(OH)₂ | 80 |
| Compound | | | | |
| LMP-420 | | | | |

As shown by Table 1, a representative compound of Formula I designated in Table 1 as LMP-420, namely the inventive 2-amino-6-chloro-9-[(5-dihydroxyboryl)-pentyl] purine, was found to be at least I 0-fold more potent than any of the other comparison compounds tested from Formula II.

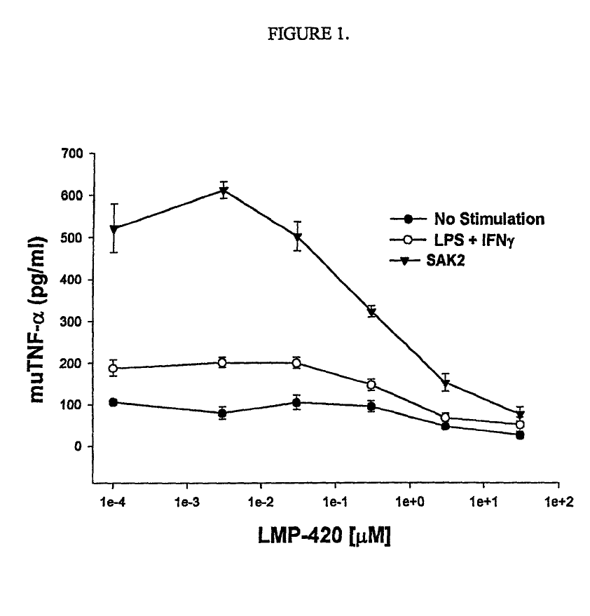

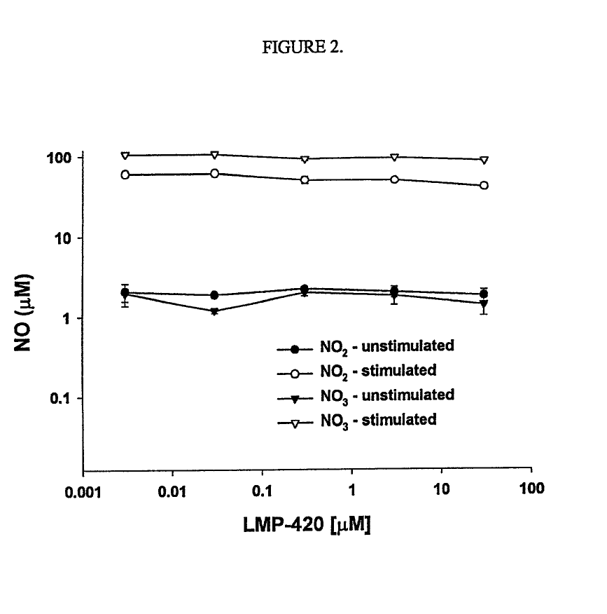

The above-mentioned inventive compound of Formula I, 2-amino-6-chloro-9-[(5-dihydroxyboryl)-pentyl] purine, labeled as LMP-420, was tested for its effect on TNF-α production by a murine macrophage cell line in accordance with the above described assay and the results are illustrated in FIG. 1. As shown in FIG. 1, LMP-420 inhibited the production of TNF-α by a cultured murine macrophage cell line with an IC₅₀ of approximately 1 μM. As shown in FIG. 2, this same compound, 2-amino-6-chloro-9-[(5-dihydroxyboryl)-pentyl] purine, had no effect on the production of nitric oxide as measured by the release of nitrate or nitrite. Nitric oxide is an important mediator for host defense against bacteria, parasites, etc. and is an important mediator in cardiovascular function. Therefore, inhibiting TNF without inhibiting nitric oxide is a very desirable attribute. The present inventors are not aware of any other inhibitors of TNF which do not inhibit also NO, thus reinforcing the uniqueness of LMP-420.

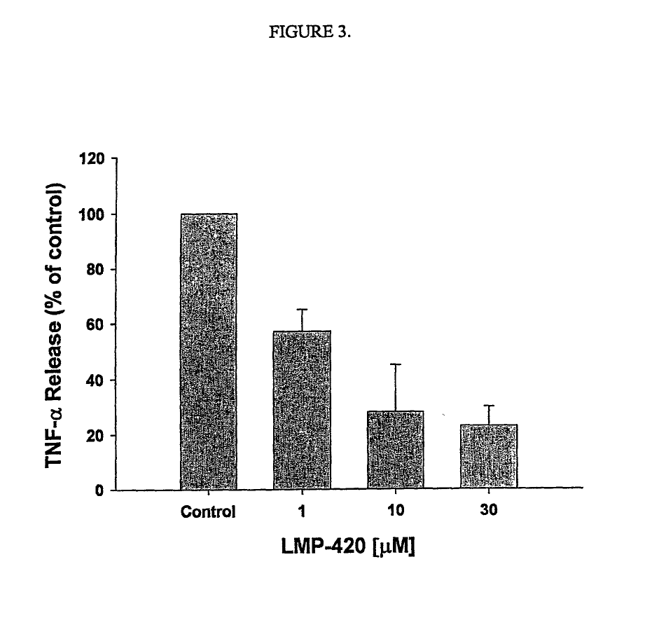

The above-mentioned inventive compound of Formula I, 2-amino-6-chloro-9-[(5-dihydroxyboryl)-pentyl] purine, labeled as LMP-420, was tested for its effects on TNF-α production by a murine mast cell line in accordance with the above described assay and the results are illustrated in FIG. 3. As shown in FIG. 3, LMP-420 inhibited the production of TNF-α by a cultured murine mast cell line with an IC₅₀ of approximately 2 to 3 μM.

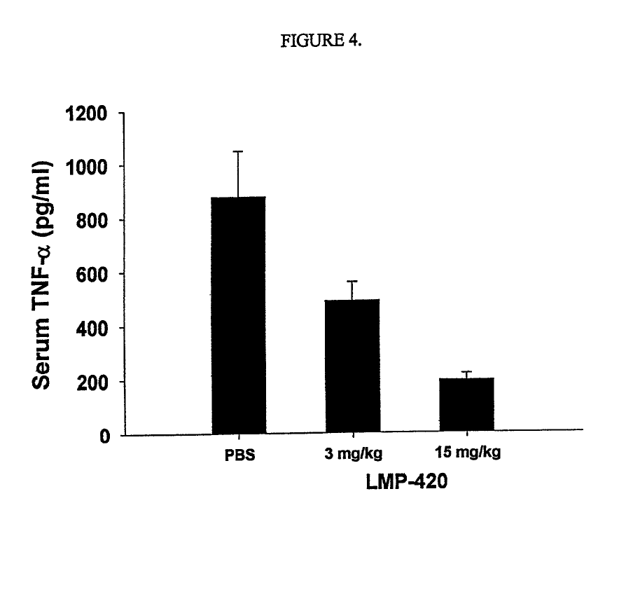

The above-mentioned inventive compound of Formula I, 2-amino-6-chloro-9-[(5-dihydroxyboryl)-pentyl] purine, labeled as LMP-420, was tested for its effects on serum TNF-α production in mice infected with Plasmodium chabaudi in accordance with the above described assay and the results are illustrated in FIG. 4. As shown in FIG. 4, LMP-420 inhibited the production of TNF-α in Plasmodium chabaudi infected mice with an IC₅₀ of approximately 3 to 4 mg/kg.

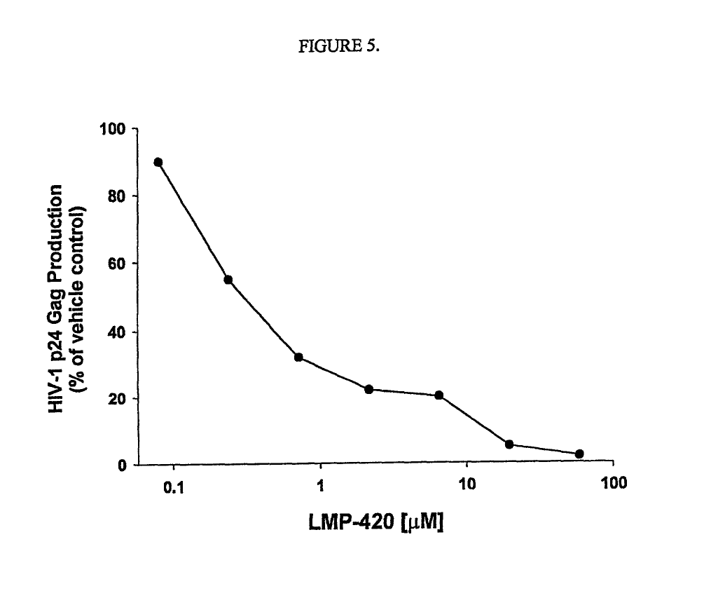

The above-mentioned inventive compound of Formula I, 2-amino-6-chloro-9-[(5-dihydroxyboryl)-pentyl] purine, labeled as LMP-420, was tested for its effects on replication of HIV-1_{Bal} (Bal is a particular strain of HIV-1) in human peripheral blood mononuclear cells in accordance with the above described assay and the results are illustrated in FIG. 5. As shown in FIG. 5, LMP-420 inhibited the replication of HIV-1_{Bal} in human peripheral blood mononuclear cells with an IC₅₀ of approximately 200 to 300 nM.

Chemokines have well characterized proinflammatory actions, including the ability to induce extravasation of leukocytes that participate in chronic inflammation. One such chemokine, RANTES, is a member of the C-C chemokine family and known to bind to C-C chemokine receptors such as CCR1 and CCR5. Studies have suggested that the chemokine receptors are prime therapeutic targets for treating inflammatory and autoimmune diseases. Thus compounds which inhibit the expression of chemokine receptors might be expected to have antiinflammatory activity. For instance, administration of a CCR1/CCR5 receptor antagonist, Met-RANTES, in a rat model of chronic colitis resulted in a significant reduction of colonic damage as well as reduced the recruitment into the colon of monocytes, mast cells, and neutrophils (Ajuebor et al, J. Immunol., 2001, 166:552-8). Another study (Rottman et al, Eur. J. Immunol., 2000, 30:2372-7) demonstrated that mice in which CCR1 was deleted were protected, at least partially, against experimental allergic encephalomyelitis (EAE), a routinely used model for multiple sclerosis (MS). Blease et al (J. Immunol., 2000, 165:1564-72) demonstrated that mice that were deficient in CCR1 had significantly fewer goblet cells and less subepithelial fibrosis around large airways in an Aspergillus fumigatus-induced allergic airway disease model of asthmatic hyperresponsiveness. There are a number of additional publications supporting the role of CCR1 and CCR5 in the pathophysiology of inflammation.

Furthermore, since CCR5 is a co-receptor for HIV-1 infection, inhibition of the expression of CCR5 might be expected to inhibit HIV-1 replication. However, inhibition of the other receptor for RANTES, CCR1, might also be expected to have an inhibitory effect on HIV-1 replication since decreased levels of CCR1 might result in increased levels of RANTES, a chemokine which has been reported to block HIV-1 replication by competing for the HIV-1 CCR5 co-receptor.

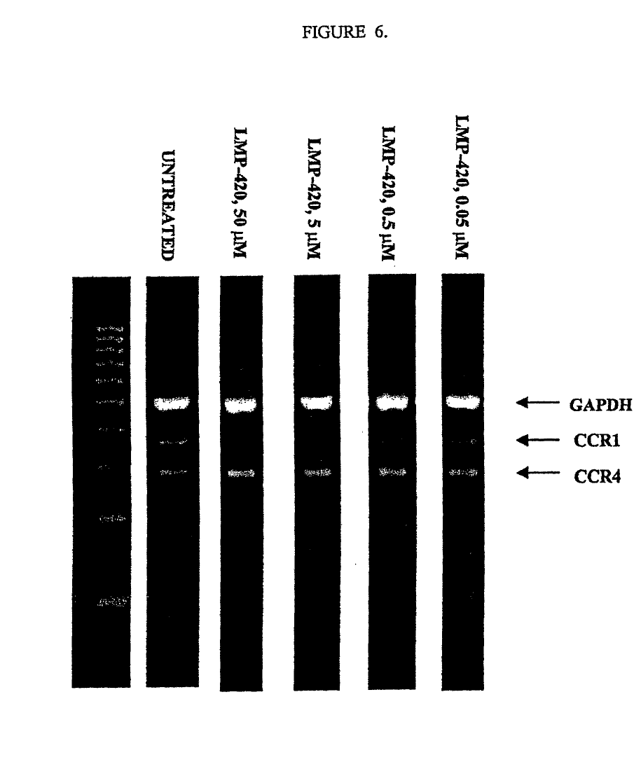

The ability of LMP-420 to affect the expression of C-C chemokine receptors on normal human PBMC was tested as illustrated in FIG. 6. As shown by FIG. 6, incubation of normal human PBMC with LMP-420 resulted in a dose-dependent inhibition of the expression of mRNA encoding for CCR1 as determined by RT-PCR analysis. There was no effect on CCR4, the only other C-C chemokine receptor whose mRNA could be detected in these unstimulated human PBMC. Also, there was no effect on the mRNA for GAPDH which is used as a “housekeeping gene” to verify that equal amounts of mRNA are being reversed transcribed for the analysis. Thus, the ability of LMP-420 to inhibit the expression of CCR1 might contribute to antiinflammatory properties of LMP-420, as well as its ability to inhibit HIV-1 replication.

It will be understood that various details of the invention may be changed without departing from the scope of the invention. Furthermore, the foregoing description is for the purpose of illustration only, and not for the purpose of limitation—the invention being defined by the claims.

What is claimed is:

1. A compound of Formula I 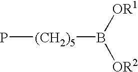
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

2. The compound of claim 1, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

3. The compound of claim 2, wherein the halogen is selected from the group consisting of Br, I, Cl, F, and combinations thereof.

4. The compound according to claim 1, wherein R¹ and R² are both hydrogen.

5. The compound of claim 1, wherein R¹ and R² together are a propylene chain.

6. The compound of claim 1, wherein the compound is 2-amino-6-chloro-9-[(5-dihydroxyboryl)-pentyl]purine.

7. A method for the treatment of a warm-blooded vertebrate animal affected with a medical condition selected from the group consisting of invasive diseases, infections, inflammatory states, and combinations thereof, said method comprising administering to the vertebrate animal a treatment effective amount of a compound of Formula I 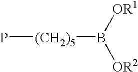
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

8. The method of claim 7, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

9. The method of claim 8, wherein the halogen is selected from the group consisting of Br, I, Cl, F, and combinations thereof.

10. The compound of claim 7, wherein R¹ and R² together are a propylene chain.

11. The method of claim 7, wherein the compound is 2-amino-6-chloro-9-[(5-5-dihydroxyboryl)-pentyl]purine.

12. The method of claim 7, wherein administering a compound of Formula I to said vertebrate animal comprises administering a compound of Formula I to a vertebrate animal selected from the group consisting of birds and mammals.

13. The method of claim 12, wherein said mammals are selected from the group consisting of humans, ruminants, carnivores, horses, and swine.

14. The method of claim 7, wherein administering an effective amount of a compound of Formula I comprises administering a compound of Formula I, where administration is selected from the group consisting of enterally, parenterally, transdermally, buccally, sublingually, orally, and a combination thereof.

15. The method of claim 14, wherein orally administering comprises administering a compound of Formula I in a form selected from the group consisting of a fluid form, tablet form, powder form, human food form, animal feed form, and combinations thereof.

16. The method of claim 15, wherein the fluid form comprises a compound of Formula I admixed in a liquid suitable therefor selected from the group consisting of water, a rehydration solution, nutritional fluid, and combinations thereof.

17. The method of claim 7, wherein administering a compound of Formula I comprises administering an effective amount of a compound of Formula I administered to the vertebrate animal in a range from about 0.05 milligram to about 2.0 milligram of the compound of Formula I per kilogram of body weight of the vertebrate animal.

18. The method of claim 7, wherein administering a compound of Formula I comprises administering to the vertebrate animal a compound of Formula I composition including an additional ingredient selected from the group consisting of an excipient, a nutriment, a carrier, a surfactant, a medicament other than a compound of Formula I, and combinations thereof.

19. The method of claim 18, wherein the medicament other than a compound of Formula I is selected from the group consisting of osmolytic polyols, osmolytic amino acids, analgesics, antibiotics, cardiotonics, electrolytes, and combinations thereof.

20. A method for the treatment of a warm-blooded vertebrate animal affected with a medical condition comprising septic shock, said method comprising administering to the vertebrate animal a treatment effective amount of a compound of Formula I 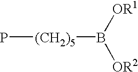
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

21. The method of claim 20, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

22. The method of claim 21, wherein the halogen is selected from the group consisting of Br, I, Cl, F, and combinations thereof.

23. The method according to claim 20, wherein R¹ and R² are both hydrogen.

24. The method of claim 20, wherein R¹ and R² together are a propylene chain.

25. The method of claim 20, wherein the compound is 2-amino-6-chloro-9-[(5-dihydroxyboryl)-pentyl]purine.

26. A method for the treatment of a warm-blooded vertebrate animal affected with a medical condition comprising rheumatoid arthritis, said method comprising administering to the vertebrate animal a treatment effective amount of a compound of Formula I 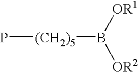
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

27. The method of claim 26, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

28. The method of claim 27, wherein the halogen is selected from the group consisting of Br, I, Cl, F, and combinations thereof.

29. The method according to claim 26, where R¹ and R² are both hydrogen.

30. The method of claim 26, wherein R¹ and R² together are a propylene chain.

31. The method of claim 26, wherein the compound is 2-amino-6-chloro-9-[(5-dihydroxyboryl)-pentyl]purine.

32. A method for the treatment of a warm-blooded vertebrate animal affected with a medical condition comprising osteoarthritis, said method comprising administering to the vertebrate animal a treatment effective amount of a compound of Formula I 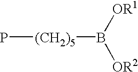
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

33. The method of claim 32, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

34. The method of claim 33, wherein the halogen is selected from the group consisting of Br, I, Cl, F, and combinations thereof.

35. The method according to claim 32, wherein R¹ and R² are both hydrogen.

36. The method of claim 32, wherein R¹ and R² together are a propylene chain.

37. The method of claim 32, wherein the compound is 2-amino-6-chloro-9-[(5-dihydroxyboryl)-pentyl]purine.

38. A method for the treatment of a warm-blooded vertebrate animal affected with a medical condition comprising ulcerative colitis, said method comprising administering to the vertebrate animal a treatment effective amount of a compound of Formula I 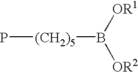
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

39. The method of claim 38, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

40. The method of claim 39, wherein the halogen is selected from the group consisting of Br, I, Cl, F, and combinations thereof.

41. The method according to claim 38, where R¹ and R² are both hydrogen.

42. The method of claim 38, wherein R¹ and R² together are a propylene chain.

43. The method of claim 38, wherein the compound is 2-amino-6-chloro-9-[(5-dihydroxyboryl)-pentyl]purine.

44. A method for the treatment of a warm-blooded vertebrate animal affected with a medical condition comprising Crohn's disease, said method comprising administering to the vertebrate animal a treatment effective amount of a compound of Formula I 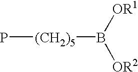
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

45. The method of claim 44, wherein P is selected from the group consisting of adenine, guanine, xanthine, and hypoxanthine and 8-substituted, 6-substituted- or 2,6-disubstituted-purines wherein the substituents are selected from the group consisting of halogen and amino.

46. The method of claim 45, wherein the halogen is selected from the group consisting of Br, I, Cl, F, and combinations thereof.

47. The method according to claim 44, wherein R¹ and R² are both hydrogen.

48. The method of claim 44, wherein R¹ and R² together are a propylene chain.

49. The method of claim 44, wherein the compound is 2-amino-6-chloro-9-[(5-dihydroxyboryl)-pentyl]purine.

50. A method for the treatment of a warm-blooded vertebrate animal affected with a medical condition comprising psoriatic arthritis, said method comprising administering to the vertebrate animal a treatment effective amount of a compound of Formula I 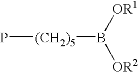
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

51. The method of claim 50, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

52. The method of claim 51, wherein the halogen is selected from the group consisting of Br, I, Cl, F, and combinations thereof.

53. The method according to claim 50, wherein R¹ and R² are both hydrogen.

54. The method of claim 50, wherein R¹ and R² together are a propylene chain.

55. The method of claim 50, wherein the compound is 2-amino-6-chloro-9-[(5-dihydroxyboryl)-pentyl]purine.

56. A method for the treatment of a warm-blooded vertebrate animal affected with a medical condition comprising psoriasis, said method comprising administering to the vertebrate animal a treatment effective amount of a compound of Formula I 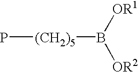
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

57. The method of claim 56, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

58. The method of claim 57, wherein the halogen is selected from the group consisting of Br, I, Cl, F, and combinations thereof.

59. The method according to claim 56, wherein R¹ and R² are both hydrogen.

60. The method of claim 56, wherein R¹ and R² together are a propylene chain.

61. The method of claim 56, wherein the compound is 2-amino-6-chloro-9-[(5-dihydroxyboryl)-pentyl]purine.

62. A method for the treatment of a warm-blooded vertebrate animal affected with a medical condition comprising chronic heart failure, said method comprising administering to the vertebrate animal a treatment effective amount of a compound of Formula I 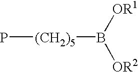
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

63. The method of claim 62, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

64. The method of claim 63, wherein the halogen is selected from the group consisting of Br, I, Cl, F, and combinations thereof.

65. The method according to claim 62, wherein R¹ and R² are both hydrogen.

66. The method of claim 62, wherein R¹ and R² together are a propylene chain.

67. The method of claim 62, wherein the compound is 2-amino-6-chloro-9-[(5-dihydroxyboryl)-pentyl]purine.

68. A method for the treatment of a warm-blooded vertebrate animal affected with a medical condition comprising congestive heart failure, said method comprising administering to the vertebrate animal a treatment effective amount of a compound of Formula I 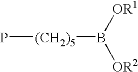
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

69. The method of claim 68, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

70. The method of claim 68, wherein the halogen is selected from the group consisting of Br, I, Cl, F, and combinations thereof.

72. The method according to claim 68, wherein R¹ and R² are both hydrogen.

72. The method of claim 68, wherein R¹ and R² together are a propylene chain.

73. The method of claim 68, wherein the compound is 2-amino-6-chloro-9-[(5-dihydroxyboryl)-pentyl]purine.

74. A method for the treatment of a warm-blooded vertebrate animal affected with a medical condition comprising asthma, said method comprising administering to the vertebrate animal a treatment effective amount of a compound of Formula I 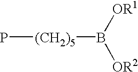
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

75. The method of claim 74, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

76. The method of claim 75, wherein the halogen is selected from the group consisting of Br, I, Cl, F, and combinations thereof.

77. The method according to claim 74, wherein R¹ and R² are both hydrogen.

78. The method of claim 74, wherein R¹ and R² together are a propylene chain.

79. The method of claim 74, wherein the compound is 2-amino-6-chloro-9-[(5-dihydroxyboryl)-pentyl]purine.

80. A method for the treatment of a warm-blooded vertebrate animal affected with a medical condition comprising multiple sclerosis, said method comprising administering to the vertebrate animal a treatment effective amount of a compound of Formula I 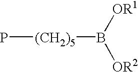
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

81. The method of claim 80, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

82. The method of claim 81, wherein the halogen is selected from the group consisting of Br, I, Cl, F, and combinations thereof.

83. The method according to claim 80, wherein R¹ and R² are both hydrogen.

84. The method of claim 80, wherein R¹ and R² together are a propylene chain.

85. The method of claim 80, wherein the compound is 2-amino-6-chloro-9-[(5-dihydroxyboryl)-pentyl]purine.

86. A method for the treatment of a warm-blooded vertebrate animal affected with a medical condition comprising non insulin-dependent diabetes mellitus, said method comprising administering to the vertebrate animal a treatment effective amount of a compound of Formula I 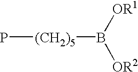
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

87. The method of claim 86, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

88. The method of claim 87, wherein the halogen is selected from the group consisting of Br, I, Cl, F, and combinations thereof.

89. The method according to claim 86, wherein R¹ and R² are both hydrogen.

90. The method of claim 86, wherein R¹ and R² together are a propylene chain.

91. The method of claim 86, wherein the compound is 2-amino-6-chloro-9-[(5-dihydroxyboryl)-pentyl]purine.

92. A method for the treatment of a warm-blooded vertebrate animal affected with a medical condition comprising atherosclerosis, said method comprising administering to the vertebrate animal a treatment effective amount of a compound of Formula I 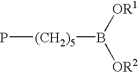
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

93. The method of claim 92, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

94. The method of claim 93, wherein the halogen is selected from the group consisting of Br, I, Cl, F, and combinations thereof.

95. The method according to claim 92, wherein R¹ and R² are both hydrogen.

96. The method of claim 92, wherein R¹ and R² together are a propylene chain.

97. The method of claim 92, wherein the compound is 2-amino-6-chloro-9-[(5-dihydroxyboryl)-pentyl]purine.

98. A method for the treatment of a warm-blooded vertebrate animal affected with a medical condition comprising multiple myeloma, said method comprising administering to the vertebrate animal a treatment effective amount of a compound of Formula I 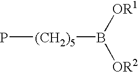
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

99. The method of claim 98, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

100. The method of claim 99, wherein the halogen is selected from the group consisting of Br, I, Cl, F, and combinations thereof.

101. The method according to claim 98, wherein R¹ and R² are both hydrogen.

102. The method of claim 98, wherein R¹ and R² together are a propylene chain.

103. The method of claim 98, wherein the compound is 2-amino-6-chloro-9-[(5-dihydroxyboryl)-pentyl]purine.

104. A method for the treatment of a warm-blooded vertebrate animal affected with a medical condition comprising neurological ischemia, said method comprising administering to the vertebrate animal a treatment effective amount of a compound of Formula I 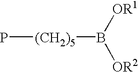
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

105. The method of claim 104, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

106. The method of claim 105, wherein the halogen is selected from the group consisting of Br, I, Cl, F, and combinations thereof.

107. The method according to claim 104, wherein R¹ and R² are both hydrogen.

108. The method of claim 104, wherein R¹ and R² together are a propylene chain.

109. The method of claim 104, wherein the compound is 2-amino-6-chloro-9-[(5-dihydroxyboryl)-pentyl]purine.

110. A method for the treatment of a warm-blooded vertebrate animal affected with a medical condition comprising Alzheimer's disease, said method comprising administering to the vertebrate animal a treatment effective amount of a compound of Formula I 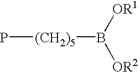
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

111. The method of claim 110, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

112. The method of claim 111, wherein the halogen is selected from the group consisting of Br, I, Cl, F, and combinations thereof.

113. The method according to claim 110, wherein R¹ and R² are both hydrogen.

114. The method of claim 1 10, wherein R¹ and R² together are a propylene chain.

115. The method of claim 110, wherein the compound is 2-amino-6-chloro-9-[(5-dihydroxyboryl)-pentyl]purine.

116. A method for the treatment of a warm-blooded vertebrate animal affected with a medical condition comprising periodontitis, said method comprising administering to the vertebrate animal a treatment effective amount of a compound of Formula I 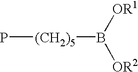
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

117. The method of claim 116, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

118. The method of claim 117, wherein the halogen is selected from the group consisting of Br, I, Cl, F, and combinations thereof.

119. The method according to claim 116, wherein R¹ and R² are both hydrogen.

120. The method of claim 116, wherein R¹ and R² together are a propylene chain.

121. The method of claim 116, wherein the compound is 2-amino-6-chloro-9-[(5-dihydroxyboryl)-pentyl]purine.

122. A method for the treatment of a warm-blooded vertebrate animal affected with a medical condition comprising malaria, said method comprising administering to the vertebrate animal a treatment effective amount of a compound of Formula I 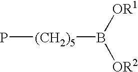
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

123. The method of claim 122, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

124. The method of claim 123, wherein the halogen is selected from the group consisting of Br, I, Cl, F, and combinations thereof.

125. The method according to claim 122, wherein R¹ and R² are both hydrogen.

126. The method of claim 122, wherein R¹ and R² together are a propylene chain.

127. The method of claim 122, wherein the compound is 2-amino-6-chloro-9-[(5-dihydroxyboryl)-pentyl]purine.

128. A method for the treatment of a warm-blooded vertebrate animal affected with a medical condition comprising celebral malaria, said method comprising administering to the vertebrate animal a treatment effective amount of a compound of Formula I 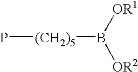
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

129. The method of claim 128, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

130. The method of claim 129, wherein the halogen is selected from the group consisting of Br, I, Cl, F, and combinations thereof.

131. The method according to claim 128, wherein R¹ and R² are both hydrogen.

132. The method of claim 128, wherein R¹ and R² together are a propylene chain.

133. The method of claim 128, wherein the compound is 2-amino-6-chloro-9-[(5-dihydroxyboryl)-pentyl]purine.

134. A method for the treatment of a warm-blooded vertebrate animal affected with a medical condition comprising AIDS, said method comprising administering to the vertebrate animal a treatment effective amount of a compound of Formula I 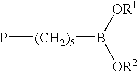
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

135. The method of claim 134, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

136. The method of claim 135, wherein the halogen is selected from the group consisting of Br, I, Cl, F, and combinations thereof.

137. The method according to claim 134, wherein R¹ and R² are both hydrogen.

138. The method of claim 134, wherein R¹ and R² together are a propylene chain.

139. The method of claim 134, wherein the compound is 2-amino-6-chloro-9-[(5-dihydroxyboryl)-pentyl]purine.

140. A method for the treatment of a warm-blooded vertebrate animal affected with a medical condition comprising cachexia associated with HIV infection, said method comprising administering to the vertebrate animal a treatment effective amount of a compound of Formula I 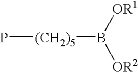
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

141. The method of claim 140, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

142. The method of claim 141, wherein the halogen is selected from the group consisting of Br, I, Cl, F, and combinations thereof.

143. The method according to claim 140, wherein R¹ and R² are both hydrogen.

144. The method of claim 140 wherein R¹ and R² together are a propylene chain.

145. The method of claim 140, wherein the compound is 2-amino-6-chloro-9-[(5-dihydroxyboryl)-pentyl]purine.

146. A method for the treatment of a warm-blooded vertebrate animal affected with a medical condition comprising cachexia associated with cancer, said method comprising administering to the vertebrate animal a treatment effective amount of a compound of Formula I 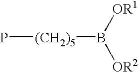
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

147. The method of claim 146, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

148. The method of claim 147, wherein the halogen is selected from the group consisting of Br, I, Cl, F, and combinations thereof.

149. The method according to claim 146, wherein R¹ and R² are both hydrogen.

150. The method of claim 146, wherein R¹ and R² together are a propylene chain.

151. The method of claim 146, wherein the compound is 2-amino-6-chloro-9-[(5-dihydroxyboryl)-pentyl]purine.

152. A method for the treatment of a warm-blooded vertebrate animal affected with a medical condition comprising cachexia associated with infection, said method comprising administering to the vertebrate animal a treatment effective amount of a compound of Formula I 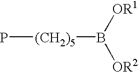
wherein R¹ and R² are both hydrogen atoms or R¹ and R² together are a 3 to 5 alkylene chain bridging the two oxygen atoms and P is a purine base residue bonded via the N⁹; and the pharmaceutically acceptable salts thereof.

153. The method of claim 152, wherein P is selected from the group consisting of adenine, guanine, xanthine, and hypoxanthine and 8-substituted-, 6-substituted- or 2,6-disubstituted-purines wherein the substituents are selected from the group consisting of halogen and amino.

154. The method of claim 153, wherein the halogen is selected from the group consisting of Br, I, Cl, F, and combinations thereof.

155. The method according to claim 152, wherein R¹ and R² are both hydrogen.

156. The method of claim 152, wherein R¹ and R² together are a propylene chain.

157. The method of claim 152, wherein the compound is 2-amino-6-chloro-9-[(5-dihydroxyboryl)-pentyl]purine.
